(19) United States
(12) Patent Application Publication
(10) Pub. No.: US 2025/0025890 A1
(43) Pub. Date: Jan. 23, 2025

(54) AIR FILTERING DEVICE FOR AN AIR CONDITIONER

(71) Applicants: MAHLE INTERNATIONAL GMBH, Stuttgart (DE); KARLSRUHER INSTITUT FÜR TECHNOLOGIE, Eggenstein-Leopoldschafen (DE)

(72) Inventors: Andrei BOLOGA, Stutensee (DE); Daniel EBERLE, Ludwigsburg (DE); Sven PETZNER, Welzheim (DE); Hans-Joachim GEHRMANN, Weingarten (DE)

(21) Appl. No.: 18/769,584

(22) Filed: Jul. 11, 2024

(30) Foreign Application Priority Data
Jul. 18, 2023 (DE) 102023206813.3

(51) Int. Cl. B03C 3/41; B03C 3/14

(52) U.S. Cl. CPC B03C 3/41; B03C 3/14

(57) ABSTRACT

An air filtering device for an air conditioner, in particular for filtering out particles, preferably pollutants, from air is provided. The air filtering device comprises a flow path through which air flows along a flow direction, an ionizer for generating ions in the flow path, and grid structure made of an electrically conductive material, that is upstream of the ionizer in the flow path in the flow direction, which forms a first electrode. A filter is downstream of the ionizer in the flow path in the flow direction which has a layer that forms a second electrode. The air filtering device also comprises a high voltage power source that has an electric pole and an electric counter-pole for generating a high voltage between the pole and the counter pole. The pole is electrically connected to the ionizer, or its discharge electrode, and the counter-pole is electrically connected to the first and second electrodes.

### CROSS-REFERENCE TO RELATED APPLICATIONS

This application claims priority from German Patent Application No. 102023206813.3,filed on Jul. 18, 2023, the entirety of which is hereby incorporated by reference herein.

The invention relates to an air filtering device for an air conditioner.

Modern air conditioners for vehicle interiors typically contain filters with which noxious substances, gases, particulates and unpleasant odors are removed from the air supplied to the vehicle interior.

The high particulate content in the vehicle environment, and thus the air conditioner environment, has proven to be problematic, resulting in high demands to the filtering of particulates from the air that is supplied to the vehicle interior. Filters with a fiber layer are used for filtering out dust particles. Because the space available for these air conditioners is normally limited in modern motor vehicles, there is also comparatively little space for such filters. To ensure that sufficient air can be supplied to the vehicle interior by the air conditioner, these filters must have a low flow resistance, such that pressure losses generated in the air also remain low. For this reason, conventional filters often have an open pore filter fiber layer for removing particles, resulting in a comparatively low mechanical capacity for dust removal on the part of the filter.

To counteract this, the removal of dust particles is improved through electrostatic charging. Unfortunately, the electrostatic charge generated in the production process diminishes over time, such that the filter quickly becomes ineffective with regard to filtering dust.

The object of the present invention is to therefore create new ways of developing air filtering devices. In particular, a better embodiment of such an air filtering device is to be obtained, which makes use of the above approach using an electrostatic charge, and results in an improved removal of particles over the entire service life of the filter.

This problem is solved by the subject matter of the independent claims. Preferred embodiments are the subject matter of the dependent claims.

The fundamental idea of the invention is to place an ionizer containing numerous discharge electrodes—also known to the person skilled in the art as corona discharge electrodes—upstream of a counter electrode, which is placed in the air flow, electrically separated from the discharge electrodes. This allows an electric field to be generated in the flow path with which particles in the air can be ionized.

The invention proposes electrically connecting a filter that can be electrostatically charged, which is downstream of the discharge electrodes in the ionizer, to the counter electrode that is upstream of the ionizer. This results in another counter electrode downstream of the ionizer in addition to the upstream counter electrode.

Consequently, an electric field is generated between the discharge electrodes in the ionizer and the counter electrodes, resulting in an improved charging of particles and a polarization effect in the filter. This also increases the long-term particle removal efficiency of the air filtering device due to the polarization effect of the filter, even when the filter is already heavily clogged with particles.

This results in an air filtering device that efficiently removes particles, in particular pollutants, from the air in the flow path over the entire service life of the air filtering device, or the filter that is used in the air filtering device.

In detail, the air filtering device according to the invention contains a flow path through which air can flow. There is an ionizer in the flow path, preferably containing discharge electrodes for generating ions therein. There is a grid made of an electrically conductive material upstream of the ionizer in the flow path, which forms a first electrode. The first electrode forms a counter electrode to the discharge electrodes and charges particles upstream of the discharge electrodes.

Downstream of the ionizer in the flow path there is a filter that has a layer forming a second electrode. This layer is made of an electrically conductive material, or comprises an electrically conductive material. The filter can preferably be in the form of a boot, thus having a bellows-like form.

The air filtering device also contains a high-voltage power source with an electric pole and counter-pole, and a counter-pole for generating a high voltage between the pole and counter-pole, preferably between −5 kV and −15 kV. The pole in the high-voltage power source is electrically connected to the ionizer, and the counter-pole in the high-voltage power source is electrically connected to the first and second electrodes.

To generate the corona discharge, a direct current with negative polarity is preferably applied, because a corona discharge with negative polarity results in greater stability and a more effective corona flow of electrons than a corona discharge with positive polarity. The electric pole of the high-voltage power source is preferably therefore the negative pole, and the electric counter-pole is the positive pole for the high-voltage power source.

Alternatively, the electric pole of the high-voltage power source can also be the positive pole, and the counter-pole can be the negative pole within the framework of the invention.

In a preferred embodiment, the filter contains a layer through which air can flow that is made of an electrically insulating material, in particular a dielectric material, for removing particles from the air, which is upstream or downstream of the layer forming the second electrode in the flow path. In particular, this second layer can be electrostatically charged. This increases the filtering effect of the second layer, and therefore the entire filter.

In an advantageous development, the electrically conductive layer of the filter forming the second electrode is formed by activated carbon, or contains activated carbon.

The two layers of the filter are ideally on top of one another in the flow path. The electric field generated with the electrically conductive layer results in the ionized particles striking the electrically insulating layer, such that they are filtered out of the air.

In an advantageous development, the grid structure contains at least two, preferably more, first rods, spaced apart from one another, which extend in the flow path in a direction transverse, preferably orthogonal, to the direction in which the air flows. In this embodiment, the grid structure contains at least two, preferably more, second grid rods, which extend in the flow path in a direction transverse, preferably orthogonal, to the the first grid rods as well as the direction in which the air flows. The grid structure forming the first electrode can be made of stainless steel wire. The grid structure can have holes of 1 mm×1 mm to 10 mm×10 mm, preferably between 3 mm×3 mm and 6 mm ×6 mm. The diameter of the wire forming the grid is to be selected such that the grid has the necessary mechanical stability. The corona discharge should also be generated without any arc-over between the grid and the sharp-edged electrode rods. There should also not be any return corona discharges from the grid.

In a preferred embodiment, the first electrode, or the grid structure, is electrically connected to the second electrode, the electrically conductive layer in the filter. Consequently, the two electrodes have the same electrical potential, which has an advantageous effect on the shape of the field lines of the electrical field generated with the two electrodes, and the polarization that is to be obtained.

The first and second electrodes can therefore be directly connected electrically to one another, such that they have the same electrical potential.

In another preferred embodiment, the ionizer contains an ionizer electrode for generating the ions in the flow path for the air. The ionizer electrode can then contain numerous discharge electrodes.

The ionizer electrode preferably contains at least one electrode rod, from which at least one electrode tip for generating the ions, preferably in the form of a needle, protrudes, preferably in the direction opposite that of the air flow. The tips of the electrodes preferably all extend in the direction opposite that of the air flow. Each electrode tip can form a separate discharge electrode in the ionizer. An extremely strong electrical field can be generated with ionizer electrodes shaped like this, with a field strength of 10 kV/mm to 40 kV/mm, which is ideal for generating numerous ions with which the particles in the air flow are charged.

In another preferred embodiment, the at least one electrode rod ideally extends in a straight line in a direction that is transverse, preferably orthogonal, to the direction in which the air flows.

In one advantageous embodiment, at least two, preferably more, electrode tips protrude from at least one electrode rod, which are preferably spaced apart in the direction in which the electrode rod extends. As a result, ions can be generated over the entire cross section of the air flow.

In another preferred embodiment, the ionizer electrode contains at least two, preferably more, preferably parallel electrode rods, which are spaced apart from one another. This also facilitates the generation of ions over the entire cross section of the air flow. The electrode tips ideally extend in the direction opposite that of the air flow.

Preferably at least two, ideally all, of the adjacent electrode rods are spaced apart in a direction orthogonal to the direction in which they extend, at a distance of 20 mm to 60 mm, preferably 25 mm to 35 mm. This ensures that the pressure drop generated by the ionizer in the air remains relatively small.

Preferably at least two, ideally all, of the adjacent electrode tips are spaced apart in a direction along the extension thereof at a distance of 1 mm to 30 mm, preferably 5 mm to 9 mm. This prevents them from causing an excessive pressure drop in the air striking or passing by the electrode rods.

The electrode tips are ideally arranged in a grid in the air flow. This results in a better shape of the field lines in the electric corona field in the air flow.

According to one advantageous embodiment, at least one of the electrode tips tapers toward the first electrode, in particular conically, ideally in the direction opposite that in which the air flows. This is ideally the case with numerous, ideally all, of the electrode tips.

The effect of the polarization of the electric field on the filter element depends on the intensity of the electric field obtained with the electrodes. Higher intensities result in greater polarization effects. Consequently, the corona discharge electrodes should be as close as possible to the entry surface of the filter. In another preferred embodiment, the distance between the ionizer, in particular the electrode rods in the ionizer, to the filter is therefore no more than 30 mm, preferably no more than 7 mm.

The grid structure, or the (first and/or second) rods thereof, is preferably made of steel, ideally stainless steel, or contains such. The ionizer, or ionizer electrodes, can also be made of steel, ideally stainless steel, or contain such, in this variation.

Further features and advantages of the invention can be derived from the dependent claims, drawings, and descriptions in reference to the drawings.

It is understood that the features specified above and described below can be used not only in the given combinations but also in other combinations or in and of themselves without abandoning the framework of the present invention.

Preferred exemplary embodiments of the invention are shown in the drawings, an shall be explained below in greater detail, in which the same reference symbols are used for identical, similar, or functionally identical components.

Therein, schematically:

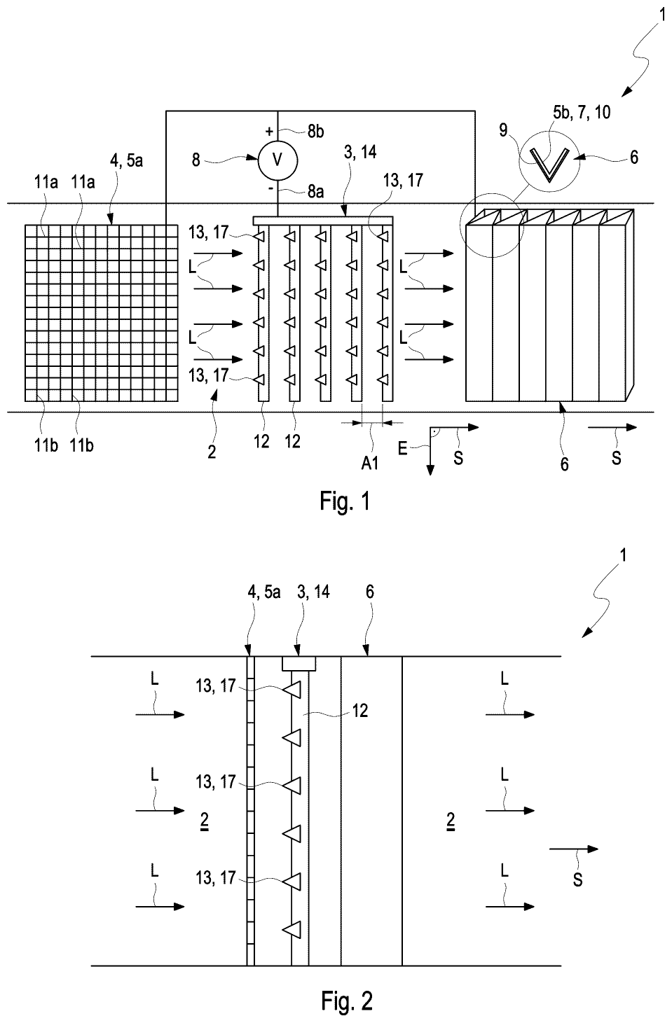

FIG. 1 shows a perspective view of an example of an air filtering device according to the invention,

FIG. 2 shows a side view of the air filtering device shown in FIG. 1, and

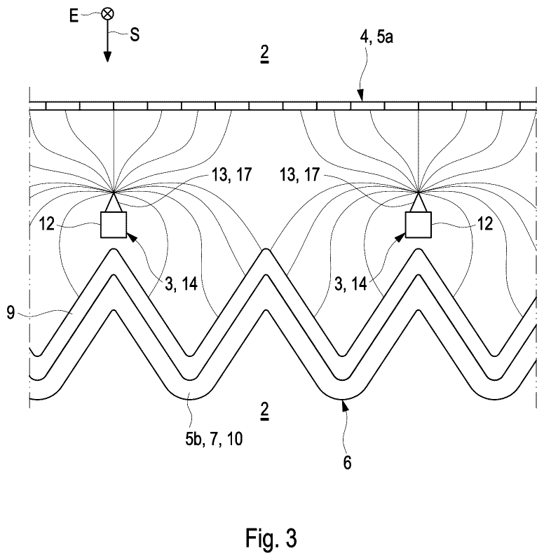

FIG. 3 shows a section of the air filtering device shown in FIGS. 1 and 2 from above.

FIG. 1 shows a perspective view of an example of an air filtering device 1 according to the invention, FIG. 2 shows a side view thereof, and FIG. 3 shows it from above. As shown in FIGS. 1 and 2, the air filtering device 1 contains a path 2, along which air L flows in a direction of flow S. The air filtering device 1 also contains an ionizer 3 in the flow path 2 for generating ions. The air filtering device 1 also contains a grid structure 4 made of an electrically conductive material that is upstream of the ionizer 3 in the flow path 2, and forms a first electrode 5a.

As can be seen in FIGS. 1 to 3, the grid structure 4 contains numerous first rods 11a, which are spaced apart and extend in the flow path 2 orthogonally to the flow direction S. The grid structure 4 also contains numerous second rods 11b, which are spaced apart and extend in the flow path 2 orthogonally to the first rods 11a and to the flow direction S. The grid structure 4, or the first and second rods 11a, 11b, can be made of steel, in particular stainless steel. The first and second rods 11a, 11b can form a plane that is perpendicular to the flow direction S.

The air filtering device 1 also contains a filter 6 that is downstream of the ionizer 3 in the flow path 2 in the flow direction S. The filter element 6 also contains a layer 7 that forms a second electrode 5b for the air filtering device 1. This layer 7 is made of an electrically conductive material in this example.

The filter 6 in the example shown in the drawings also contains a layer 9 through which the air L can flow, made of an electrically insulating material for removing particles from the air L. This layer 9 is upstream of the layer 7 forming the second electrode 5b in the flow path 2. The layer 7 of the filter 6 forming the second electrode 6b is formed by activated carbon 10. The two layers 7, 9 of the filter 6 can be layered in the flow direction S.

The air filtering device 1 also contains a high voltage power source 8 with an electric pole 8a and an electric counter-pole 8b for generating a high voltage between the pole 8a and counter-pole 8b. This voltage can be between 5 kV and 15 kV between the pole 8a and counter-pole 8b. The pole 8a is preferably a negative pole, and the counter-pole 8b is preferably positive. This can also be reversed, such that the pole 8a is positive, and the counter-pole 8b is negative.

The negative pole 8a is electrically connected to the ionizer 3 and the positive counter-pole 8b is electrically connected to the first and second electrodes 5a, 5b in FIG. 1. The ionizer 3 contains an ionizer electrode 14 comprising discharge electrodes 17 for generating ions. The ionizer electrode 14 comprises numerous electrode rods 12 that are spaced apart from one another, which have tips 13 in the form of needles that extend from each rod 12 toward the first electrode 5a, from which the ions can be generated for charging particles. The electrode tips 13 form the discharge electrodes 14. The electrode tips 13 extend from the respective rods 12 toward the grid structure 4 in the direction opposite the air flow S.

As can be seen in FIGS. 1 to 3, the electrode tips 13 are arranged in a grid in the flow path 2, and also taper conically toward the first electrode 5a. The ionizer 3, or ionizer electrode 14, and its rods 12 can be made of steel, e.g. stainless steel. The individual rods 12 each extend in the flow path 2 in a straight line in the same direction E, which is orthogonal to the flow direction S. The distance between the electrode rods 12 in the ionizer and the filter 6 is no more than 30 mm, preferably no more than 7 mm.

By way of example, all of the adjacent electrode rods 12 are spaced apart at a distance A1 in a direction that is orthogonal to the direction of extension E, which is 15 mm to 60 mm, preferably 25 to 35 mm. In this example, all of the electrode tips 13 are spaced apart at a distance along the direction of extension E of 1 mm to 30 mm, preferably 5 mm to 9 mm.

The specification can be readily understood with reference to the following Representative Paragraphs:
Representative Paragraph 1. An air filtering device (1) for an air conditioner, in particular for filtering particles, preferably pollutants, from air (L), containing
   a flow path (2) through which air (L) can flow in a flow direction(S),
   an ionizer (3), preferably containing numerous discharge electrodes (17), for generating ions in the flow path (2),
   a grid structure (4) made of an electrically conductive material, which is upstream of the ionizer (3) in the flow path (2) in the flow direction(S), and forms a first electrode (5a),
   a filter (6) downstream of the ionizer (3) in the flow path in the flow direction(S) that has a layer (7) forming a second electrode (5b), comprising an electrically conductive material or made of an electrically conductive material,
   a high voltage power source (8) containing an electric pole (8a) and an electric counter-pole (8b), for generating a high voltage (HV), in particular between 5 kV and 15 kV between the pole (8a) and counter-pole (8b),
   wherein the pole (8a) is electrically connected to the ionizer (3), and the counter-pole (8b) is electrically connected to the first and second electrodes (5a, 5b).
Representative Paragraph 2. The air filtering device according to Representative Paragraph 1, characterized in that the
   electric pole (8a) is negative, and the electric counter-pole (8b) is positive in the high voltage power source (8), or
   electric pole (8a) is positive, and the electric counter-pole (8b) is negative in the high voltage power source (8).
Representative Paragraph 3. The air filtering device according to Representative Paragraph 1 or 2, characterized in that the filter (6) comprises a layer (9) made of an electrically insulating material through which air can flow for removing particles from the air (L), which is upstream or downstream of the layer (7) forming the second electrode (5) in the flow path.
Representative Paragraph 4. The air filtering device according to Representative Paragraph 3, characterized in that the layer (7) forming the second electrode (5b) in the filter (6) is formed by activated carbon (10) or comprises activated carbon (10).
Representative Paragraph 5. The air filtering device according to any of the preceding Representative Paragraphs, characterized in that
   the grid structure (4) comprises at least two, preferably more, first rods (11a) that are spaced apart from one another, which extend in the flow path (2) in a direction transverse, preferably orthogonal, to the flow direction(S),
   the grid structure (4) comprises at least two, preferably more, second rods (11b) that are spaced apart from one another, which extend in the flow path (2) in a direction transverse, preferably orthogonal, to both the first rods (11a) and the flow direction(S).
Representative Paragraph 6. The air filtering device according to any of the preceding Representative Paragraphs, characterized in that the first electrode (5a), or the grid structure (4) is electrically connected to the second electrode (5b).
Representative Paragraph 7. The air filtering device according to any of the preceding Representative Paragraphs, characterized in that the grid structure (4) and the filter (6) are electrically connected to one another.
Representative Paragraph 8. The air filtering device according to any of the preceding Representative Paragraphs, characterized in that the ionizer (3) contains an ionizer electrode (14) for generating the ions in the flow path (2).
Representative Paragraph 9. The air filtering device according to any of the preceding Representative Paragraphs, characterized in that the ionizer electrode (14) has at least one electrode rod (12), from which at least one electrode tip (13) extends toward the first electrode (5a) to generate the ions.
Representative Paragraph 10. The air filtering device according to Representative Paragraph 9, characterized in that the at least one electrode rod (12) extends in a straight line, in a direction of extension (E) that is transverse, preferably orthogonal, to the flow direction(S) in the flow path (2).
Representative Paragraph 11. The air filtering device according to Representative Paragraph 8 or 9, characterized in that
   at least two, preferably numerous, electrode tips (13) protrude from at least one electrode rod (12), which are preferably spaced apart from one another along the direction of extension (E) of the electrode rod (12),
   wherein at least one electrode tip (13), preferably all, electrode tips (13) extend in the direction opposite the flow direction(S).
Representative Paragraph 12. The air filtering device according to any of the preceding Representative Paragraphs, characterized in that the ionizer electrode (14) comprises at least two, preferably numerous, electrode rods (12) that are spaced apart from one another, which preferably parallel to one another.
Representative Paragraph 13. The air filtering device according to Representative Paragraph 12, characterized in that at least two, preferably more, ideally all, adjacent electrode rods (12) are spaced apart from one another at a distance (A1) in a direction orthogonal to the direction of extension (E), which is 15 mm to 60 mm, preferably 25 mm to 35 mm.
Representative Paragraph 14. The air filtering device according to any of the Representative Paragraphs 11 to 13, characterized in that at least two, preferably more, ideally all, adjacent electrode tips (13) are spaced apart from one another along the direction of extension (E) at a distance of 1 mm to 30 mm, preferably 5mm to 9 mm.
Representative Paragraph 15. The air filtering device according to any of the Representative Paragraphs 11 to 14, characterized in that the electrode tips (13) are arranged in the manner of a grid in the flow path (2).
Representative Paragraph 16. The air filtering device according to any of the Representative Paragraphs 11 to 15, characterized in that at least one, preferably more, ideally all, of the electrode tips (13) taper toward the first electrode (5a), in particular conically, preferably in the direction opposite the flow direction(S).
Representative Paragraph 17. The air filtering device according to any of the preceding Representative Paragraphs, characterized in that the distance between the ionizer (3), in particular the electrode rods (12) in the ionizer (3) and the filter (6) is no more than 30 mm, preferably no more than 7 mm.
Representative Paragraph 18. The air filtering device according to any of the preceding Representative Paragraphsc, characterized in that
   the grid structure (4), or the (first and/or second) rods (11a, 11b), is made of steel, in particular stainless steel, or contains steel, and/or
   the ionizer (4), or the ionizer electrode (14), is made of steel, in particular stainless steel, or contains steel, in particular stainless steel.

1. An air filtering device for an air conditioner, in particular for filtering particles, preferably pollutants, from air, containing
a flow path through which air can flow in a flow direction,
an ionizer, containing numerous discharge electrodes (17), for generating ions in the flow path,
a grid structure formed from an electrically conductive material, the grid structure being positioned upstream of the ionizer in the flow path in the flow direction, and forms a first electrode,
a filter positioned downstream of the ionizer in the flow path in the flow direction, the filter has a layer forming a second electrode, comprising an electrically conductive material or formed from an electrically conductive material,
a high voltage power source containing an electric pole and an electric counter-pole, configured to generate a high voltage, in particular between 5 kV and 15 kV between the pole and counter-pole,
wherein the pole is electrically connected to the ionizer, and the counter-pole is electrically connected to the first and second electrodes.

2. The air filtering device according to claim 1, wherein the
electric pole is negative, and the electric counter-pole is positive in the high voltage power source, or
electric pole is positive, and the electric counter-pole is negative in the high voltage power source.

3. The air filtering device according to claim 1, wherein the filter comprises a layer formed from an electrically insulating material through which air can flow for removing particles from the air, the layer formed from the electrically insulating material is positioned either upstream or downstream of the layer forming the second electrode in the flow path.

4. The air filtering device according to claim 3, wherein the layer forming the second electrode in the filter is formed by activated carbon or comprises activated carbon.

5. The air filtering device according claim 1, wherein
the grid structure comprises at least two first rods that are spaced apart from one another, which extend in the flow path in a direction transverse to the flow direction(S),
the grid structure comprises at least two second rods that are spaced apart from one another, which extend in the flow path in a direction transverse, to both the first rods and the flow direction.

6. The air filtering device according to claim 1, wherein the first electrode, or the grid structure is electrically connected to the second electrode.

7. The air filtering device according to claim 1, wherein the grid structure and the filter are electrically connected to one another.

8. The air filtering device according to claim 1, wherein the ionizer contains an ionizer electrode for generating the ions in the flow path.

9. The air filtering device according to claim 1, wherein the ionizer electrode has at least one electrode rod, from which at least one electrode tip extends toward the first electrode to generate the ions.

10. The air filtering device according to claim 9, wherein the at least one electrode rod extends in a straight line, in a direction of extension that is transverse to the flow direction in the flow path.

11. The air filtering device according to claim 8, wherein
at least two electrode tips protrude from at least one electrode rod, which are spaced apart from one another along the direction of extension of the electrode rod,
wherein at least one electrode tip extends in a direction opposite the flow direction

12. The air filtering device according to claim 1, wherein the ionizer electrode comprises at least two electrode rods that are spaced apart from one another.

13. The air filtering device according to claim 12, wherein at least two adjacent electrode rods are spaced apart from one another at a distance in a direction orthogonal to the direction of extension, which is 15 mm to 60 mm.

14. The air filtering device according to claim 11, wherein at least two adjacent electrode tips are spaced apart from one another along the direction of extension at a distance of 1 mm to 30 mm.

15. The air filtering device according to claim 11, wherein the electrode tips are arranged in the manner of a grid in the flow path.

16. The air filtering device according to claim 11, wherein at least one of the electrode tips taper toward the first electrode.

17. The air filtering device according to claim 1, wherein the distance between the lar the electrode rods in the ionizer and the filter is no more than 30 mm.

18. The air filtering device according to claim 1, wherein
the grid structure, or the first rod and/or the second rod rods, comprises at least in part steel, and/or
the ionizer, or the ionizer electrode, comprises at least in part steel.

19. The air filtering device of claim 5, wherein the at least two first rods extend in an orthogonal flow path to the first direction, and the at least two second rods extend in an orthogonal direction to both the first rods and the flow direction.

20. The air filtering device of claim 12, wherein the at least two electrode rods are parallel to one another.

21. The air filtering device of claim 13, wherein all of the adjacent electrode rods are spaced apart from one another in the direction orthogonal to the direction of extension at a distance of 25 mm to 35 mm.

22. The air filtering device of claim 14, wherein all of the adjacent electrode tips are spaced apart from one another along the direction of extension at a distance of 5 mm to 9 mm.

23. The air filtering device of claim 16, wherein all of the electrode tips taper toward the first electrode conically and in a direction opposite the flow direction.

24. The air filtering device of claim 17, wherein the distance between the electrode rods in the ionizer and the filter is no more than 7 mm.

25. The air filtering device of claim 10, wherein the at least one electrode extends in a direction that is orthogonal to the flow direction in the flow path.
